(19) United States
(12) Patent Application Publication
(10) Pub. No.: US 2006/0173073 A1
(43) Pub. Date: Aug. 3, 2006

(54) Microfine zinc-glycerol complexes

(75) Inventors: Reginald M Taylor, Sommerton Park (AU); Margaret L Taylor, Sommerton Park (AU); David P Fairlie, Springwood (AU); Robert M Moffitt, Los Angeles, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Pure Pharamaceuticals Inc., Santa Monica, CA (US)

(21) Appl. No.: 10/524,252

(22) PCT Filed: Jul. 9, 2003
(86) PCT No.: PCT/US03/21271

§ 371 Date: Mar. 7, 2006

(30) Foreign Application Priority Data
Jul. 9, 2002 (US) 60394240

(51) Int. Cl. A61K 31/315

(52) U.S. Cl. 514/494

(57) ABSTRACT

The present invention relates in part to a method of manufacturing zinc-glycerol complexes and the zinc-glycerol complexes so produced, which unexpectedly results in microfine zinc-glycerol complex particles which have a mean particle length (i.e., major axis) of less than about 1.0 micron. The zinc-glycerol complex particles of the invention also generally maintain a hexagonal crystalline structure, thereby retaining superior lubricity and tactile properties. The invention also provides pharmaceutical and/or therapeutic compositions comprising microfine zinc-glycerol complex particles, and methods for using such compositions.

### FIELD OF THE INVENTION

The present invention relates to the field of inorganic chemistry. More particularly, the present invention relates to zinc-glycerol complexes, methods for its manufacture, and its use in pharmaceutical and cosmetic formulations.

### BACKGROUND OF THE INVENTION

Zinc glycerolate, or Zinc (1,2,3-Propanetriolato(2-)-O1,O2) Homopolymer, Stereoisomer, also known as zinc monoglycerolate or glycerato zinc or Glyzinc®, CAS Registry Number 87189-25-1, empirical formula (C₃H₆O₃Zn)ₓ, is a crystalline zinc-glycerol complex defined by its lubricous and tactile properties, which arise from the unique insoluble plate-like, two-dimensional structure of its generally hexagonal polymeric crystals. Several methods are known in the art for the formation of zinc-glycerol complexes. However, the morphology of the zinc-glycerol complex resulting from these known methods varies as a function of both synthesis technique (i.e., crystal growth) and post-synthesis milling/grinding (i.e., overall particle dimensions). Such variations in morphology greatly influence the physical, chemical, and biochemical properties of the zinc-glycerol complex.

For instance, Radoslovich et al (Austral. J. Chem. 23 (1970) 1963-1970) first disclosed that crystalline metal-glycerol complexes are formed by heating certain metal oxides, hydroxides or salts with glycerol at temperatures above 110° C. The metals include cobalt, iron, manganese and zinc, and the complexes formed differ from those previously obtained by reaction in aqueous solution at room temperature in that they are insoluble in water and organic solvents. The zinc compound reportedly is obtained from zinc carbonate at 160° C., from zinc acetate at 110° C. to 160° C., and from zinc hydroxide at 200° C. but no experimental data is provided. It is also reported that slow heating of the reaction mixture produced well developed crystals at 110° C. but that higher temperatures are required to obtain smaller plates. The particle size and tactility of these products is not reported, however, it was generally assumed at the time that temperatures of at least 120° C. were required to obtain zinc glycerolate (as that term is used herein).

Fairlie et al (Agents and Actions 36 (1992) 152-158) refers to the use of zinc to treat local skin disorders and some forms of systemic inflammatory disease, including arthritis. Reference is made in the opening paragraph of the Introduction to forming Glyzinc by heating zinc oxide with glycerol at 200° C. to 300° C. In the exemplified process, a dispersion of zinc hydroxide in ethanol is heated with glycerol to 120° C. until ethanol and absorbed water evaporates and then the temperature is raised to 240° C.-275° C. for one hour to expel water.

Taylor et al (U.S. Pat. No. 4,544,761) discloses the use of zinc glycerolate for cosmetic, prophylactic and therapeutic purposes. Specific reference is made to its use in treating ammoniacal dermatitis in babies (diaper rash) and pruritus; for alleviating psoriasis; for preventing fungal or bacterial decomposition of tissue and the resulting odors, especially in tinea pedis; for preventing industrial dermatitis; and for treating ichthyosis. The zinc glycerolate is specified to be of 10 to 100 micrometer particle size and to be the reaction product of zinc oxide (or a zinc oxide precursor compound) with glycerol at 120° C. to 300° C. It is stated that the reaction of zinc oxide with glycerol proceeds slowly below 200° C., but very rapidly above 220° C. The only specified reactant ratio of zinc compound to glycerol is 1:10.

The preferred methods of preparing zinc glycerolate specified in Taylor are (a) heating a mixture of zinc oxide (or zinc oxide precursor compound) with glycerol at a temperature of about 260° C.; (b) adding zinc oxide (or zinc oxide precursor compound) to glycerol at 120° C.; and then increasing the temperature of the mixture to about 260° C.; and (c) adding zinc oxide (or zinc oxide precursor compound) to glycerol at 220° C., and then increasing the temperature of the mixture to about 260° C. Process (a) is exemplified in Example 4 using zinc oxide; process (b) is exemplified in Example 2 using zinc oxide; process (c) is exemplified in Examples 1 and 3 using zinc oxide and zinc acetate respectively. In all four Examples, the mixture was heated at 260° C. for 1 hour. Crude zinc glycerolate is separated from the reaction mixture by filtering and then washing first with ethanol and then with acetone before being dried.

WO 87/01281 discloses the percutaneous, subcutaneous and intramuscular as well as oral administration of zinc glycerolate. It specifically refers to the use of zinc glycerolate in the treatment of diabetes; as an antimicrobial or antibacterial agent; for the treatment of zinc deficiency; as an anti-inflammatory agent, especially in the treatment of arthritis and psoriasis; and as a gastroprotective agent, especially in the treatment of gastric ulcers. As in Taylor, described above, it is specified that the zinc glycerolate is the reaction product of the zinc oxide (or a zinc oxide precursor compound) with glycerol at 120° C. to 300° C. There is no exemplification of the manufacture of zinc glycerolate.

WO 87/01379 discloses the use of zinc glycerolate as a UV-shielding material and bonding agent in rubbers and plastics. It is specified that the zinc glycerolate is prepared by mixing zinc oxide (or a zinc oxide precursor compound) with glycerol at a temperature of about 260° C., and, after cooling, pouring the mixture into water, filtering, washing and drying. It is stated that the reaction will proceed at lower (unspecified) temperatures more slowly. The only specified reactant ratio of zinc compound to glycerol is 1:10. There is no exemplification of the manufacture of zinc glycerolate.

Taylor (U.S. Pat. No. 4,943,316) discloses the preparation of zinc glycerolate by exposing a solution or suspension of a zinc compound in glycerol to microwave radiation. An object of the invention is to avoid the high temperatures required in the convention process. The exemplified zinc compounds are zinc acetate and zinc hydroxide but there is no exemplification of the manufacture of zinc glycerolate.

Apisariyakulm et al (The Medical Journal of Australia 152 (1990) 54) discloses the use of zinc glycerolate in the treatment of oral herpetic sores.

WO 93/02130 discloses a flexible gas-permeable polymeric film incorporating zinc glycerolate for use in the modified atmosphere packaging of fresh produce, especially flowers, fruit and vegetables, to extend the post harvest life thereof. No reference is made to the process by which the zinc glycerolate is prepared.

Bos (U.S. Pat. No. 5,475,123) disclose a process for the preparation of zinc glycerolate comprising reaction substantially stoichiometric amounts of a zinc compound (e.g., zinc oxide or zinc acetate) and a polyhydroxy compound (e.g., glycerol) in the presence of a catalyst (e.g., an acid or acid salt) at a temperature ranging from about 120° C. to about 250° C. Bos further reports that the particles produced according to the disclosed method are subjected to a size reduction step to reduce the particle size to less than approximately 25 microns, preferably 9 microns. However, such mechanical size reduction distorts the hexagonal crystalline structure of the zinc glycerolate, which in turn can distort the physical, chemical and biochemical properties of the substance.

Matkin et al (U.S. Pat. No. 5,646,324) discloses that zinc glycerolate can be prepared from zinc oxide (or a zinc oxide precursor compound) at temperatures below 110° C., and that the zinc glycerolate thus obtained can be separated from the reaction mixture by centrifuging. Matkin further reports that glycerol is removed from the residue by simultaneously or alternately washing with C₁-C₄ alkanol, especially isopropanol, and centrifuging.

Each of these methods for producing zinc-glycerol complexes disclosed in the prior art, and the compositions so produced exhibit widely variable morphologies which thereby limits their ability to be used for pharmaceutical or therapeutic applications. As such, there remains a need in the art for improved zinc-glycerol complex compositions and methods for their manufacture which improved and reproducible particle morphologies.

### SUMMARY OF THE INVENTION

Accordingly, the present invention relates in part to a method of manufacturing zinc-glycerol complexes, particularly zinc glycerolate, and the complexes so produced, which unexpectedly results in microfine zinc-glycerol complex particles which have a mean particle length (i.e., major axis) of less than about 1.0 micron.

The zinc-glycerol complex particles of the invention also generally maintain a hexagonal crystalline structure, thereby retaining superior lubricity and tactile properties.

The present invention also provides compositions comprising a plurality of microfine zinc-glycerol complex particles, and therapeutic methods for using such compositions.

Additional advantages and features of the present invention will be apparent from the following detailed description, drawings and examples which illustrate preferred embodiments of the invention.

### BRIEF DESCRIPTION OF THE DRAWINGS

FIGS. 1-12 are representative SEM micrographs of microfine zinc glycerolate produced according to the invention.

FIGS. 13-16 illustrate particle chunkiness distribution vs. particle length for microfine zinc glycerolate produced according to the invention and comparative commercially available samples of zinc glycerolate.

### DETAILED DESCRIPTION OF THE PREFERRED EMBODIMENTS

The present invention is directed to compositions useful in a variety of pharmaceutical and/or therapeutic applications, wherein the compositions comprise zinc-glycerol complexes of a controlled morphology, particularly zinc glycerolate particles having a mean particle length (i.e., major axis) of less than about 1.0 micron. Methods for the manufacture of such zinc-glycerol complexes are also provided.

By way of background, the morphology of a substance influences the properties of the substance, including its physical, chemical and biochemical properties. For example, smaller particles can exhibit improved lubricity on the skin due to their ability to fit into smaller crevices in the skin. Additionally, smaller particles generally provide finer texture and appear more transparent by passing visible light. Smaller particles can also have a higher surface-area-to-volume ratio, providing relatively increased exposure to the surrounding environment for a more rapid chemical reaction or drug availability. Further, particle packing can be aided by a narrow particle size distribution. As such, it is desirable to control the morphology of particulate compositions in a manner which results in a relatively narrow particle size distribution of smaller particle sizes.

A. Zinc-Glycerol Complexes

The present invention can be used to form microfine zinc-glycerol complexes which are insoluble plate-like, two-dimensional structures of generally hexagonal polymeric crystals. In a preferred embodiment, the zinc-glycerol complexes are zinc glycerolate.

According to the present invention, it was unexpectedly discovered that zinc-glycerol complexes can be formed which exhibit a controlled morphology such that the mean particle length and mean particle thickness of the zinc-glycerol complex particles are less than about 1.0 micron without the need for after-synthesis grinding or milling. It was also discovered that the hexagonal crystalline structure of the zinc-glycerol complexes can be preserved, thereby resulting in a composition with improved physical, chemical, and biochemical properties.

In a preferred embodiment of the invention, the particles of the zinc-glycerol complexes are microfine, meaning that they have a reproducible particle size distribution such that at least about 75 percent of the particles have a particle length (i.e., major axis) of between about 0.20 microns and about 1.25 microns; at least about 50 percent of the particles have a particle length of between about 0.20 microns and about 0.75 microns; and at least about 25 percent of the particles have a particle length of between about 0.20 microns and about 0.60 microns. Preferably, the microfine zinc-glycerol complexes of the invention comprise no more than about 20 percent, preferably no more than about 10 percent, and most preferably no more than about 5 percent of zinc-glycerol complex particles having a particle length above about 1.0 μm. Microfine zinc-glycerol complex particles having a mean particle length of between about 0.70 and 1.0 μm and below are most preferred.

B. Methods of Manufacturing Microfine Zinc-Glycerol Complexes

The invention also provides a novel technique for producing microfine zinc-glycerol complexes, wherein the complexes so produced exhibit reproducible particle sizes smaller than those previously observed. Generally, the method involves reacting a salt form of zinc with an excess of glycerol under controlled temperature, pressure, and agitation for a time sufficient to effectuate reaction between the dissolved zinc and the glycerol. More particularly, it was unexpectedly discovered that temperature alone is not the driving factor of particle size. Rather, without being limited by theory, it is believed that the overall rate of excitement (e.g., temperature, pressure, and/or rate of agitation) leading to molecular intermingling of the starting materials is the driving factor of particle size. Further, the rate of excitement tends to correlate inversely with the time of reaction, meaning that a lower-temperature reaction would require a longer reaction time.

The salt form of zinc can be any form which is soluble in glycerol, and is preferably zinc acetate. The reaction temperature preferably ranges from about 110° C. to about 180° C. at atmospheric pressure (760 mmHg). As would be recognized by one of ordinary skill in the art, the preferred temperature range would vary depending on the reaction pressure. For example, a temperature of 110° C. at 760 mmHg would be equivalent to a temperature of about 50° C. at 100 mmHg and a temperature of about 95° C. at 500 mmHg.

The overall ratio of glycerol to zinc in the starting material preferably ranges from 2:1 glycerol:zinc to about 10:1 glycerol:zinc, more preferably from about 3:1 glycerol:zinc to about 4:1 glycerol:zinc.

The reaction mixture is preferably agitated at a rate sufficient to create shear forces within the reaction mixture such that there is appropriate intermingling of the reaction starting materials. In one embodiment, a mechanical blade stirrer set to between about 1000 rpm and about 1500 rpm, preferably between about 1300 rpm and about 1400 rpm, may be used.

The reaction time depends on the rate of excitement, but generally ranges from about 1 hour to about 5 hours, preferably from about 2 hours to about 4 hours.

In one embodiment, the glycerol is heated on an oil bath to desired temperature under the desired pressure, and the zinc salt is added to the heated glycerol. The mixture is then maintained at the desired temperature with stirring for the desired reaction time. Alternatively, the zinc salt may be slowly added to the glycerol while the reaction mixture is heating or the glycerol may be slowly added to the zinc salt to dissolve the zinc salt and then heated to effectuate the reaction.

After the reaction is complete, the reaction mixture may be cooled and washed with an appropriate solvent such as isopropanol. The washed reaction product precipitate can then be collected via, e.g., filtration or centrifugation, and dried.

C. Pharmaceutical and Therapeutic Applications

The invention also provides pharmaceutical and/or therapeutic compositions comprising the zinc-glycerol complexes of the invention. The pharmaceutical and/or therapeutic compositions of the invention can be used to treat a variety of topical conditions and can be formulated in any manner known in the art such as an ointment or powder. Additionally, formulated ointments or powders can be included in consumer products such as medicated bandages or diapers, or can be used as a lubricant in latex or nitrile gloves. In another embodiment, the zinc-glycerol complexes of the invention can be formulated as a dietary supplement to deliver the complexed zinc to a patient in need thereof. Such a dietary supplement may be in the form of a transdermal patch, or may be formulated as a tablet or pill for ingestion by the patient. In yet another embodiment, the zinc-glycerol complexes of the invention may be used as an anti-microbial or anti-bacterial agent.

Topical conditions which may be treated using the pharmaceutical and/or therapeutic compositions of the invention include inflammation, such as dermatitis (eczema), diaper rash, poison ivy/poison oak, and psoriasis; infection, such as herpes (genital, chicken pox, shingles, etc), impetigo, tinea (ringworm, athlete's foot, jock-itch, etc.), acne, and yeast (Candida, thrush, vaginal, etc.); minor wounds, such as burns, cuts, scraps, decubitus ulcers (pressure sores), hemorrhoids, and sunburn; and lip care conditions such as dry lips, chapped lips, weather damaged lips, and cold sores.

Dietary supplements including the zinc-glycerol complexes of the invention can also be used correct zinc deficiencies, such as those associated with anorexia nervousa (zinc deficiency).

In a preferred embodiment, the pharmaceutical and/or therapeutic compositions of the invention comprise a therapeutically effective amount of the zinc-glycerol complex of the invention. Preferred concentrations of the zinc-glycerol complex in the pharmaceutical and/or therapeutic compositions of the invention are in the range of about 10 to about 100 percent (wt/wt). A more preferred range of concentrations is from about 25 to about 100 percent, and even more preferred concentrations are from about 40 to about 100 percent. In particular, an amount selected form the group consisting of about 10, about 25, about 40; about 99 and about 100 percent (wt/wt) is preferred.

As used herein, the term “therapeutically effective amount” means an amount of the zinc-glycerol complex of the invention that is sufficient to show a meaningful patient benefit, i.e., healing or amelioration of chronic conditions, a reduction in inflammation or other symptoms, or an increase in rate of healing of such conditions.

Therapeutic efficacy and toxicity of the compositions may be determined by standard pharmaceutical, pharmacological, and toxicological procedures in cell cultures or experimental animals. For example, numerous methods of determining ED₅₀ (the dose therapeutically effective in 50 percent of the population) and LD₅₀ (the dose lethal of 50 percent of the population) exist. The dose ratio between therapeutic and toxic effects is the therapeutic index, and it can be expressed as the ratio ED₅₀/LD₅₀. Compositions exhibiting high therapeutic indices are preferred. The data obtained from cell culture assays or animal studies may be used in formulating a range of dosages for human use. The dosage is preferably within a range of concentrations that includes the ED₅₀ with little or no toxicity, and may vary within this range depending on the dosage form employed, sensitivity of the patient, and the route of administration.

The pH of the pharmaceutical and/or therapeutic compositions of the invention is preferably between about 6 and about 8, and may be adjusted as desired for the particular use so long as hydrolysis is avoided as known in the art. Purified water USP and various acids and bases suitable for topical use, or combinations of acids and bases, may be used for adjusting the pH of the compositions. Non-limiting examples of acids and bases include acetic acid, boric acid, citric acid, lactic acid, phosphoric acid, hydrochloric acid, sodium hydroxide, sodium phosphate, sodium borate, sodium citrate, sodium acetate, sodium lactate, and TRIS.

Additional components of the pharmaceutical and/or therapeutic compositions of the invention may be chosen from any of those used in or capable of being used in a pharmaceutical formulation, especially those designed for topical administration. A non-exclusive list of components includes preservatives, stabilizers, chelating agents, dyes, antibiotics, antimicrobials, and anti-fungal agents. Preservatives such as benzalkonium chloride may be used in a range between about 0.001 to 1 percent by weight, or any value in this range. The pharmaceutical and/or therapeutic compositions of the invention of the present invention may further comprise pharmaceutically acceptable carriers, excipients, gels, solutions, or diluents suitable for topical administration, and may include pharmaceutically acceptable polymeric suspension agents. Suitable carriers or excipients include, but are not limited to, calcium carbonate, calcium phosphate, various sugars, starches, cellulose derivatives, gelatin, and polymers such as polyethylene glycol. Suitable techniques for the formulation and administration of the compositions of the present invention may be found in Remington's Pharmaceutical Sciences, 18^{th} edition (1990).

The physical and chemical characteristics of the compositions of the invention may be modified or optimized according to the skill in the art. Thus, pH, viscosity, and the content of various additional components may be chosen from any appropriate range known or modified from the examples given here. In general, the pharmaceutical and/or therapeutic compositions of the invention may be manufactured in a manner that is itself known, e.g., by means of conventional mixing, dissolving, emulsifying, encapsulating, entrapping, or lyophilizing processes.

In yet another embodiment of the invention, the prophylactic and/or therapeutic treatment of diseases and conditions described above are provided comprising administering a pharmaceutical and/or therapeutic composition of the invention to a patient in need of such treatment. As recognized by one of skill in the art, the duration of prophylactic and therapeutic treatment will vary depending on the particular disease or condition being treated in that some conditions lend themselves to acute treatment whereas others require long-term therapy.

Application of the teachings of the invention to a specific problem or environment is within the capabilities of one having ordinary skill in the art in light of the teachings contained herein. Examples of the products and processes of the present invention appear in the following examples, which are provided by way of illustration, and are not intended to be limiting of the present invention.

### EXAMPLE 1

### Manufacture of Microfine Zinc Glycerolate

A. Mechanical Stirring, 120° C., 4:1 Glycerol:Zinc (Sample A)

Procedure: 30 g ground Zn(OAc)2.2H2O and 52 g glycerol (4 equivalents) were heated in a 120° C. oil bath (110° C. internal temperature) in a 500 mL round-bottomed flask. The mixture was stirred mechanically with an overhead stirrer, set at 1300 rpm. The size of the stainless steel blade was 7 cm. The reaction went through several stages. The initially viscous solution gradually became less viscous with heating. Water and acetic acid were given off during the reaction. The mixture became less viscous and eventually formed a clear solution, with all of the zinc acetate dissolved. The product formed rapidly after this stage, with a corresponding increase in viscosity. The mixture was heated for a total of three hours. A small portion of the reaction mixture was filtered through a high porosity frit and then washed with isopropanol and ether. This small aliquot was used for scanning electron microscopy. The reaction mixture was transferred to centrifuge tubes and was spun at 4500 rpm for 30 minutes. The clear supernatant was removed and fresh isopropanol was added and the white solid resuspended. Centrifugation and removal of the supernatant was followed by the addition of further isopropanol and the solution was then filtered through a sintered glass funnel of high porosity.

B. Mechanical Stirring, 120° C., 4:1 Glycerol:Zinc (Sample B)

Procedure: 30.0 g of (mortar and pestle) ground Zn(OAc)₂.2H2O (BDH) and 52 g of glycerol (BDH) (4:1 ratio glycerol to zinc) were placed into a 500 mL round-bottomed flask and immersed into an oil bath, which was heated over 30 minutes to an oil bath temperature of 120° C. During this time the reaction was stirred with a mechanical overhead blade stirrer (7 cm×1-2 cm) set to 1400 rpm. When the internal temperature of the reaction reached about 95° C. water was being given off. After 60 mins the solution became clear due to complete dissolution of the zinc salt (the reaction solution was at a temperature of 110° C, at this stage), and 10 mL EtOH was added portionwise after the first hour to wash down the sides of the flask. Over the next 15 mins a white solid precipitated. The reaction was clearly over within this 15 minute period. Nevertheless the reaction was maintained at this temperature with continued stirring until 3 hours after the reaction had been initiated. The flask was then removed from the oil bath, 100 mL isopropanol was added to the warm solution, which was filtered whilst still hot through a sintered glass funnel (initially gravity filtered then with vacuum suction). The reaction mixture was slow to filter. Alternatively the solution can be centrifuged (10000 rpm, 30 mins). The white precipitate was washed with two 25 mL portions of isopropanol and then washed with ether. The white solid was dried in a vacuum desiccator. Yield=15.94 g microfine zinc glycerolate.

C. Mechanical Stirring, 130° C., 4:1 Glycerol:Zinc (Sample C)

The above reaction was repeated under exactly the same conditions, except for the temperature of the oil bath being kept at 130° C. The internal temperature at the stage of product formation was 118° C.

D. Mechanical Stirring, 130° C. 3:1 Glycerol:Zinc (Sample D)

A reaction was carried out using three equivalents of glycerol at 130° C. 1.50 g ground Zn(OAc)₂.2H₂O and 1.86 g glycerol (3 equivalents) were placed in to a 10 mL round-bottomed flask. The reaction was stirred with a magnetic stirrer. The reaction was heated in an oil bath heated to 130° C. The white-colored precipitate was collected by filtration and washed with isopropanol and ether before being dried under vacuum. Yield=0.735 g microfine zinc glycerolate.

### EXAMPLE 2

### Characterization of Zinc Glycerolate

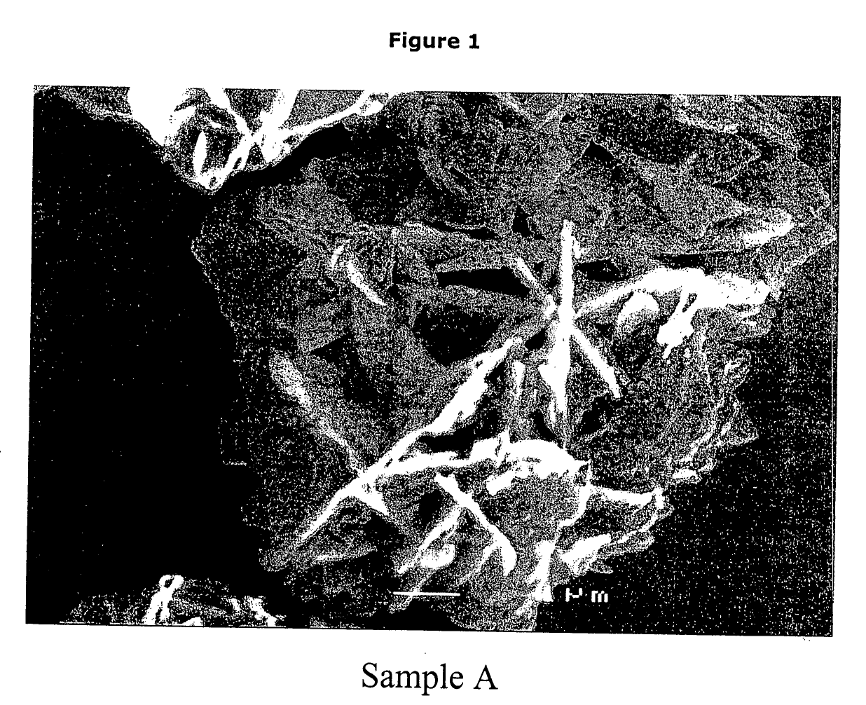

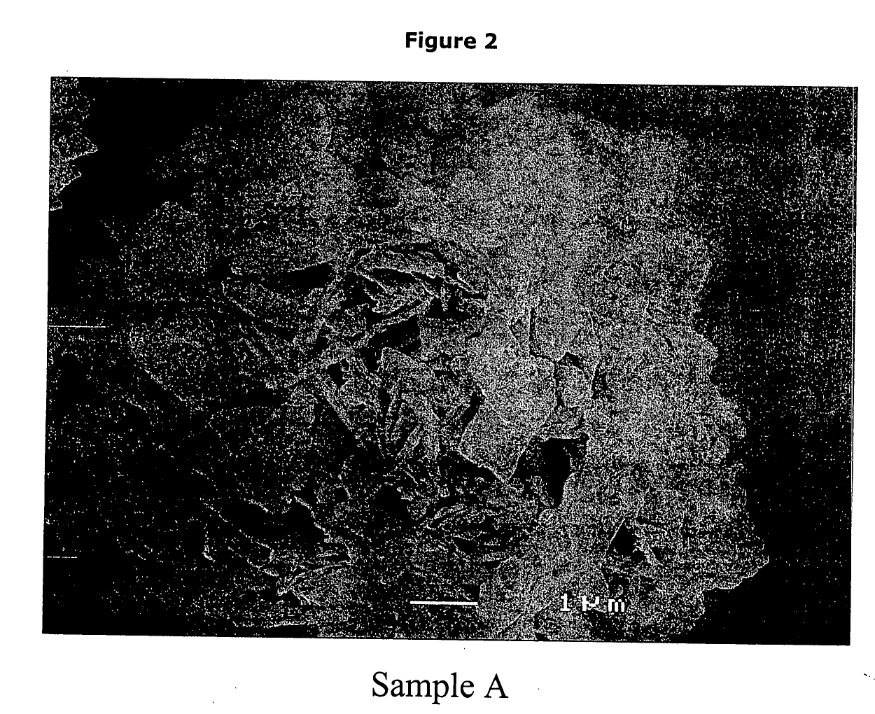

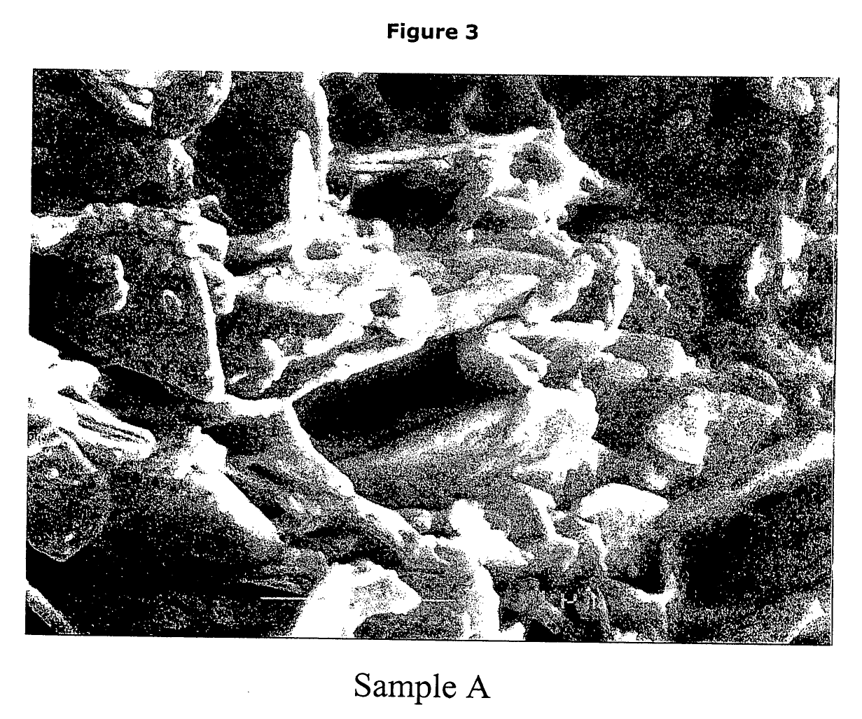

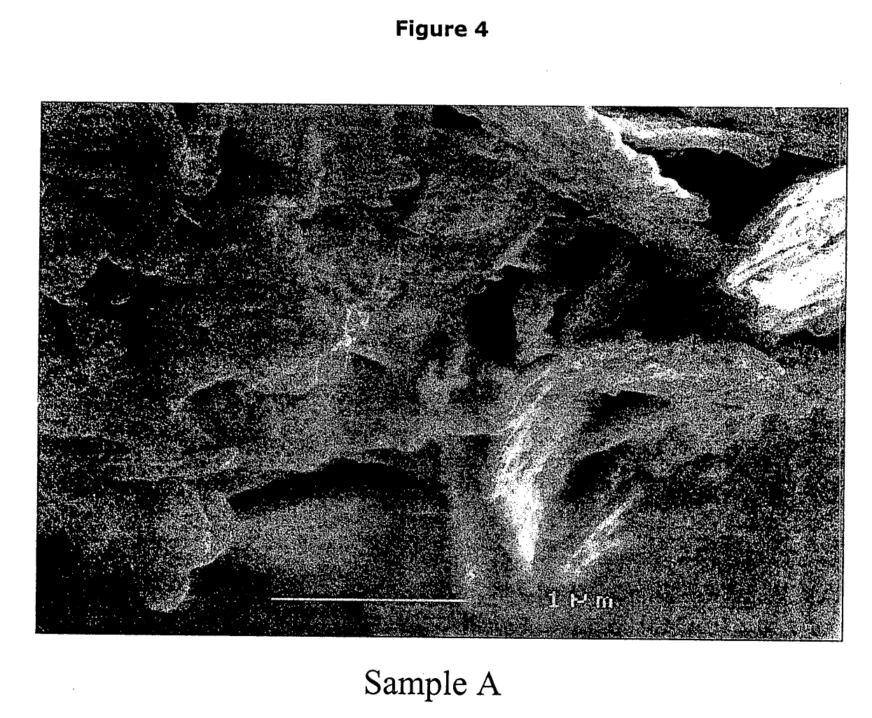

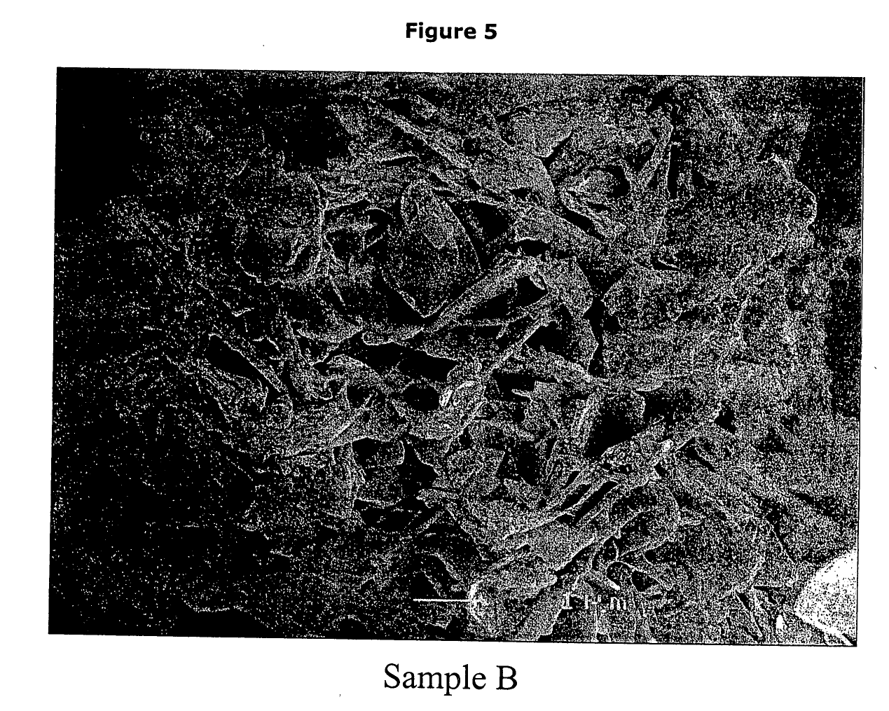

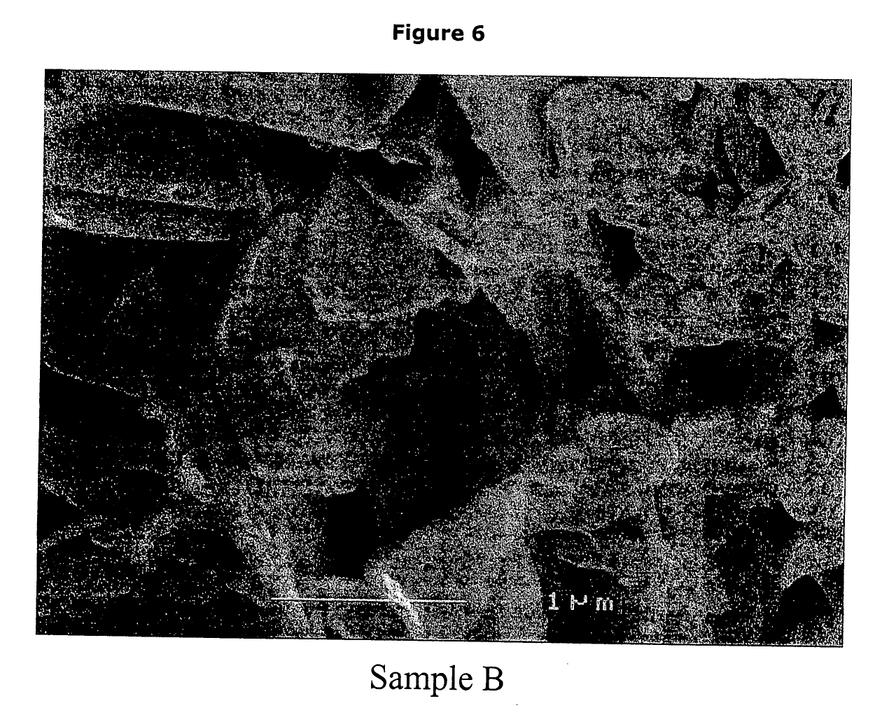

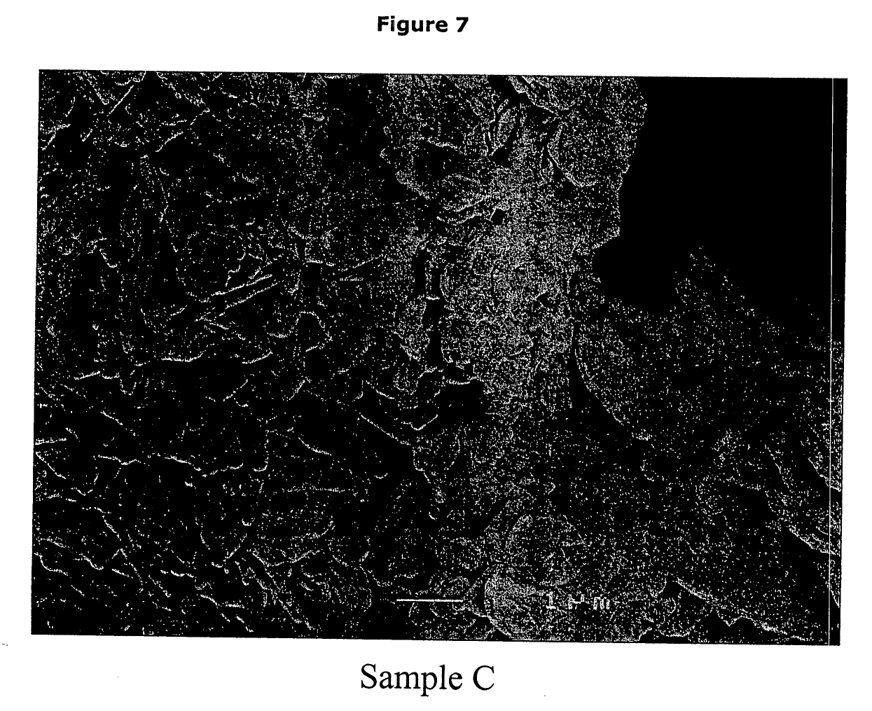

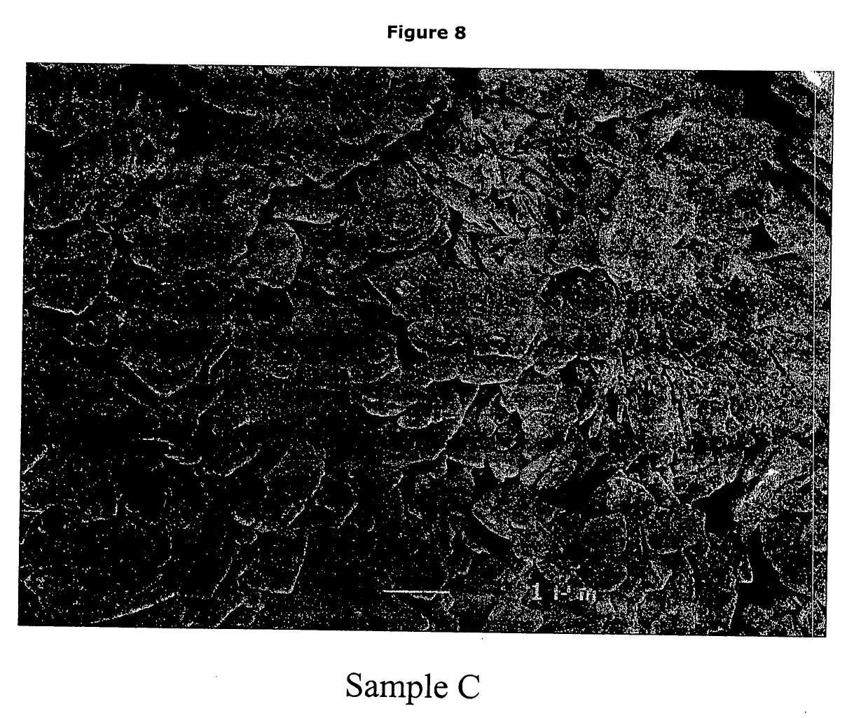

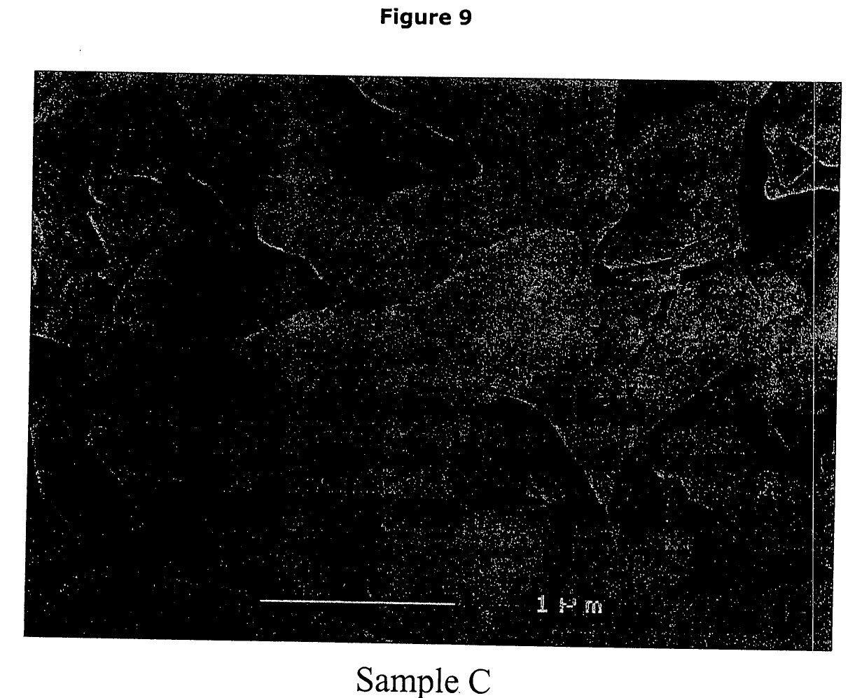

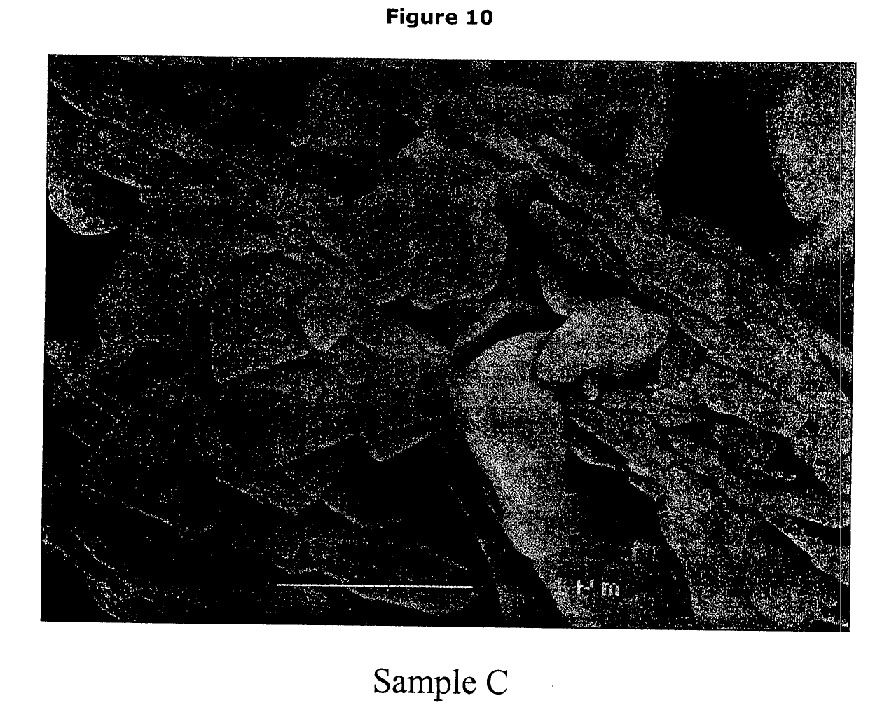

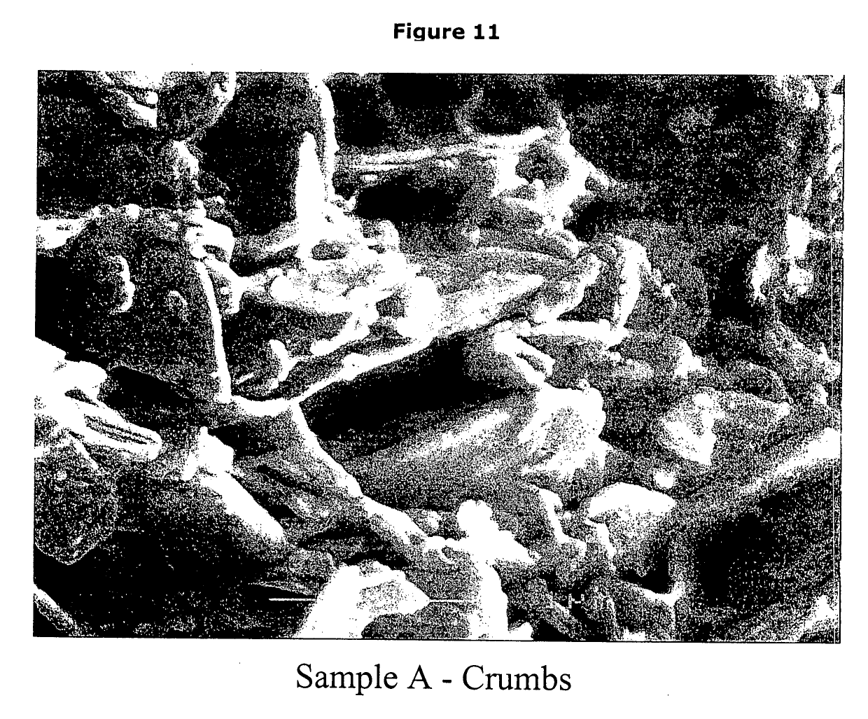

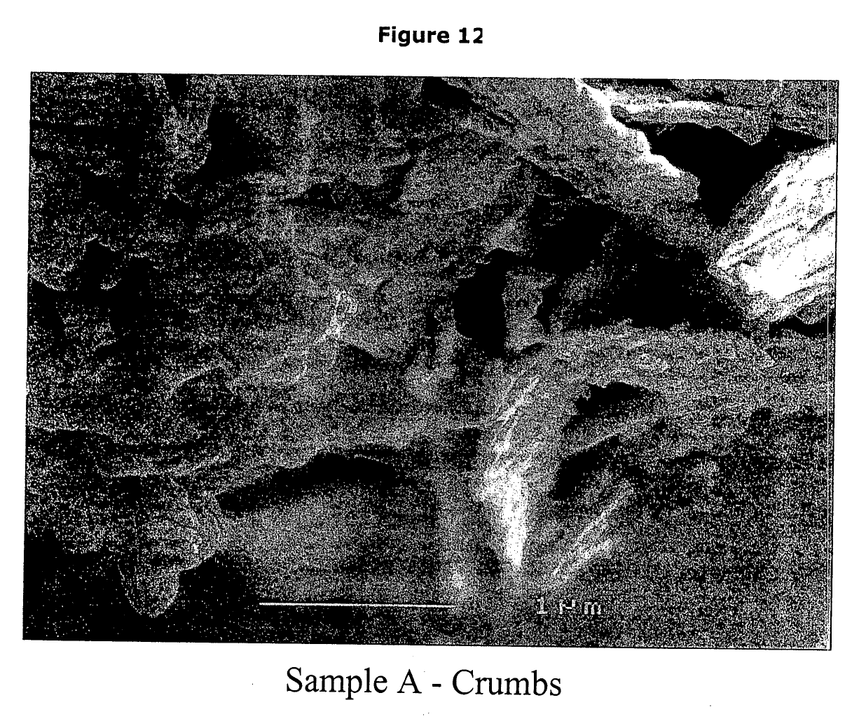

Microfine zinc glycerolate samples A, B, and C prepared at 110° C. and 118° C. (120 and 130° C. oil bath temperature respectively) as described above were examined by Scanning Electron Microscope (SEM). All samples consisted of clusters made up of smaller particles. These clusters were rounded in shape. It is believed that these clusters have formed through electrostatic attraction under the SEM conditions, this being a normal phenomenon for particles less than 5 μm in size. The 120° C. sample consisted of clusters of fairly uniform diameter of between about 20 to about 30 μm. The 130° C. sample has clusters that range in size from about 14 to about 21 μm. At higher magnification, small crumb-like particles can be seen adhering to larger plate-like particles. Generally, the particles have a thickness ranging from about 0.1 μm to about 1 μm, with a mean of about 0.8 μm. FIGS. 1-12 are representative micrographs of the SEM analysis illustrating particle clusters, particle length (i.e., major axis), particle breadth (i.e., minor axis), particle thickness, and crumb dimensions.

A. Particle Length (i.e. Major Axis)

Tables 1 and 2 summarize the results of the SEM analysis (indicated sizes are particle length). For comparison, commercially available samples of zinc glycerolate were also analyzed by SEM. Comparative Sample A was obtained from Pharmaserve (see, e.g., Matkin et al., supra). Comparative Sample B was obtained from Micronizer (see, e.g., Bos, supra). Table 3 provides a more detailed analysis of the commercially available

TABLE 1

| | Sample B | Sample C | Comp. A | Comp. B |
|---|---|---|---|---|
| Number of Particles | 26 | 85 | | |
| Minimum Size (μm) | 0.2667 | 0.125 | | |
| 25^{th} Percentile | 0.60 | 0.44 | | |
| Median (μm) | 0.70 | 0.625 | 7.143 | 1.500 |
| 75^{th} Percentile | 1.07 | 0.88 | | |
| Maximum Size (μm) | 3.1 | 1.9 | | |
| Mean | 0.9531 | 0.7194 | 7.825 | 1.460 |
| Std. Deviation | 0.6026 | 0.3921 | 4.291 | 0.4209 |
| Std. Error | 0.1182 | 0.04253 | 0.8258 | 0.8100 |

TABLE 2

| | Sample A - | Sample A - |
|---|---|---|
| | Large Particles | Crumbs |
| Number of particles | 103 | 92 |
| Minimum size (μm) | 0.6133 | 0.05333 |
| 25^{th} Percentile | 1.3 μM | 0.09 μM |
| Median (μm) | 1.9 | 0.14 |
| 75^{th} Percentile | 2.6 μM | 0.2 μM |
| Maximum Size (μm) | 5.5 μM | 0.53 μM |
| Mean | 2.0 μM | 0.16 μM |
| Std. Deviation | 0.93 | 0.08 |
| Std. Error | 0.09 | 0.008 |

TABLE 3

| Particle Length | | Comp. A | Comp. B |
|---|---|---|---|
| 0-1 | μm | 0% | 18.5% |
| 1-2 | μm | 7.4% | 74% |
| 2-5 | μm | 22.2% | 7.4% |
| Over 5 | μm | 70.4% | 0% |

B. Particle “Chunkiness”

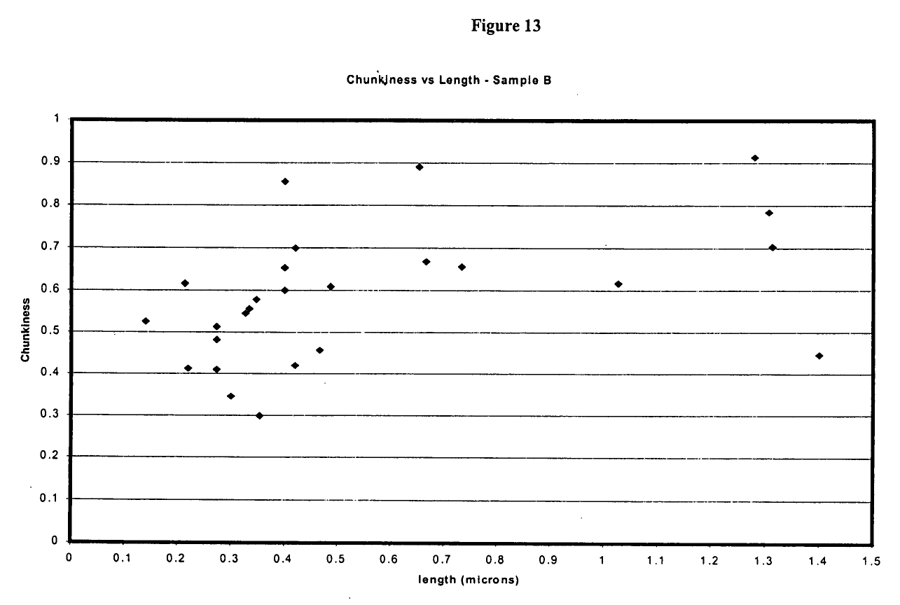

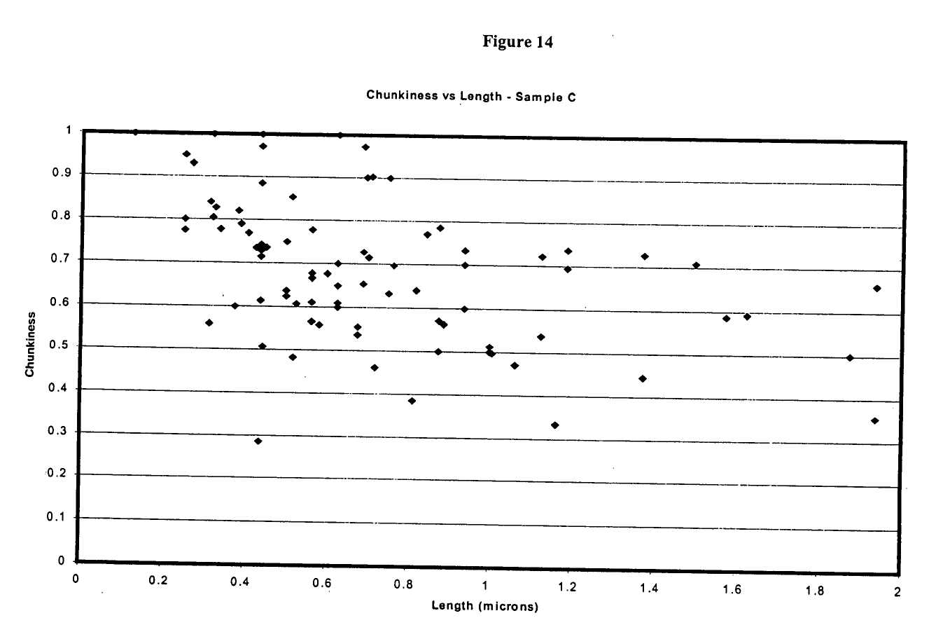

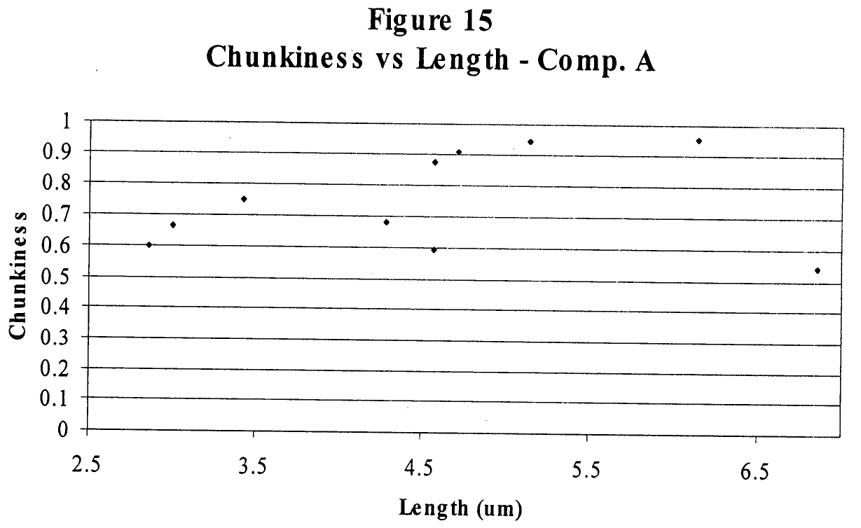

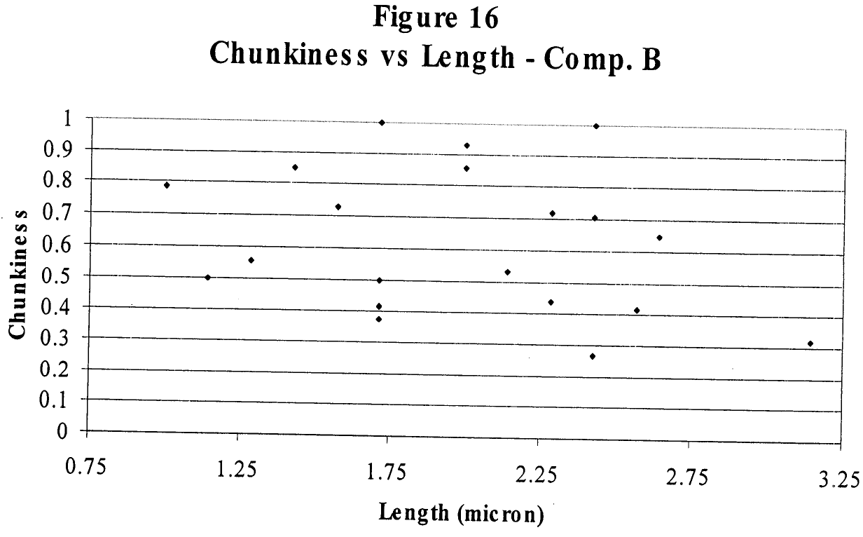

In addition to particle length, the two dimensional shape of the microfine zinc glycerolate particles produced according to Example 1 was analyzed. As an indicator of two dimensional shape, the particle “chunkiness” was determined. Particle chunkiness is the breadth of the particle (i.e., minor axis) divided by the length of the particle (i.e., major axis). Lower chunkiness values indicate that the particles are more rod or string-like. FIGS. 13 and 14 illustrate the distribution of particle chunkiness vs. particle length for Samples B and C, respectively. For comparison, FIGS. 15 and 16 illustrate the distribution of particle chunkiness vs. particle length for Comparative Samples A and B, respectively.

All publications and patents mentioned in the above specification are herein incorporated by reference. The above description, drawings and examples are only illustrative of preferred embodiments that achieve the objects, features and advantages of the present invention. It is not intended that the present invention be limited to the illustrative embodiments. Any modification of the present invention which comes within the spirit and scope of the following claims should be considered part of the present invention.

What is claimed is:

1. A composition comprising a plurality of zinc glycerolate particles, wherein said plurality of zinc glycerolate particles have a mean particle length of between about 0.70 microns and about 1.0 microns.

2. The composition of claim 1, wherein said plurality of zinc glycerolate particles have a median particle length of between about 0.60 microns and about 0.75 microns.

3. The composition of claim 1, wherein at least about 75% of said plurality of zinc glycerolate particles have a particle length of between about 0.80 microns and 1.1 microns.

4. The composition of claim 1, wherein at least about 25% of said plurality of zinc glycerolate particles have a particle length of between about 0.4 microns and about 0.6 microns.

5. The composition of claim 1, wherein said zinc-glycerolate particles are present in said composition in an amount of between about 1% and about 50% by weight of the total weight of the composition.

6. The composition of claim 1, further comprising at least one pharmaceutically acceptable carrier.

7. The composition of claim 6, wherein said plurality of zinc glycerolate particles are present in said composition in an amount of between about 1% and about 50% by weight of the total weight of the composition.

8. A pharmaceutical composition comprising a plurality of zinc glycerolate particles, wherein said plurality of zinc glycerolate particles have a mean particle length of between about 0.70 microns and about 1.0 microns, and a pharmaceutically acceptable carrier.

9. The pharmaceutical composition of claim 8, wherein said plurality of zinc glycerolate particles are present in the composition in a therapeutically effective amount for the treatment or prevention of a topical disease or condition selected from the group consisting of: eczema, diaper rash, poison ivy, poison oak, psoriasis, genital herpes, chicken pox, shingles, impetigo, tinea, acne, Candida, thrush, burns, cuts, scraps, decubitus ulcers, hemorrhoids, sunburn, dry lips, chapped lips, weather damaged lips, and cold sores.

10. A topical composition comprising a plurality of zinc glycerolate particles, wherein said plurality of zinc glycerolate particles have a mean particle length of between about 0.70 microns and about 1.0 microns, and
a pharmaceutically acceptable carrier suitable for application of the topical composition to skin as a therapeutic, prophylactic, or cosmetic substance.

11. A pharmaceutical composition for use in treating or preventing skin disorders or diseases in a mammal by topical administration to the skin, comprising a therapeutically effective amount of zinc glycerolate particles, wherein said zinc glycerolate particles have a mean particle length of between about 0.70 microns and about 1.0 microns, and
a pharmaceutically acceptable carrier suitable for topical administration.

12. A skin protectant comprising about I % to about 40 % by weight of zinc glycerolate particles, wherein said zinc glycerolate particles have a mean particle length of between about 0.70 microns and about 1.0 microns,
and a pharmaceutically acceptable carrier.

13. A method for treating topical diseases or conditions in a patient in need of such treatment, comprising topically administering to the skin a composition comprising a therapeutically effective amount of zinc glycerolate particles, wherein said plurality of zinc glycerolate particles have a mean particle length of between about 0.70 microns and about 1.0 microns.

14. The method of 13, wherein the patient is a human.

15. The method of 13, wherein the method for treating topical diseases or conditions is a method for treating herpetic diseases.

16. A method for preventing topical diseases or conditions in a patient susceptible to developing topical diseases or conditions, comprising topically administering to the skin of said patient a composition comprising a therapeutically effective amount of zinc glycerolate particles and a pharmaceutically acceptable carrier
wherein said zinc glycerolate particles have a mean particle length of between about 0.70 microns and about 1.0 microns.

17. A method for providing temporary protection to skin of a patient in need of such treatment, comprising topically administering to the skin a composition comprising a therapeutically effective amount of zinc glycerolate particles
wherein said zinc glycerolate particles have a mean particle length of between about 0.70 microns and about 1.0 microns.

18. A method for treating fever blisters and cold sores in a human in need of such treatment, comprising topically administering a composition comprising between about 1 percent and about 40 percent w/w of zinc glycerolate particles and a pharmaceutically acceptable carrier suitable for topical application of the composition;
wherein said zinc glycerolate particles have a mean particle length of between about 0.70 microns and about 1.0 microns.
